(19) United States
(12) Patent Application Publication
(10) Pub. No.: US 2016/0068867 A1
(43) Pub. Date: Mar. 10, 2016

(54) METHOD AND APPARATUS FOR CONTINUOUS FLOW BIO-FUEL PRODUCTION

(71) Applicant: TOMMY MACK DAVIS, SPARTANBURG, SC (US)

(72) Inventor: TOMMY MACK DAVIS, SPARTANBURG, SC (US)

(21) Appl. No.: 14/942,171

(22) Filed: Nov. 16, 2015

Related U.S. Application Data
(63) Continuation of application No. 13/584,336, filed on Aug. 13, 2012, now abandoned.
Application No. 13/584,336 is a continuation of application No. 12/462,951, filed on Aug. 12, 2009, now abandoned.

(51) Int. Cl. C12P 7/06; C12Q 3/00

(52) U.S. Cl. CPC C12P 7/06; C12Q 3/00

(57) ABSTRACT

A continuous flow system for production of bio-fuels using microbial cultures is provided. The present invention does not utilize batch type production, but follows a continuous flow protocol that eliminates much downtime inherent in conventional bio-fuel production systems while greatly reducing space and equipment requirements. Production is enhanced via controlled program of aeration for microbial growth and anaerobic conditions to ensure fermentation efficiency. As the system becomes more tolerant of alcohol content, efficiency increases. Feedstocks include, but are not limited to, material normally discarded from food production facilities including drink syrups, juices or waste water from corn or sugar processing plants.

### CROSS REFERENCES TO RELATED APPLICATION

THIS APPLICATION IS A CONTINUATION OF U.S. patent application Ser. No. 13/584,336 FILED Aug. 13, 2012, CURRENTLY PENDING, WHICH IS A CONTINUATION OF U.S. patent application Ser. No. 12/462,951 FILED Aug. 12, 2009, ABANDONED, INCORPORATED HEREIN BY REFERENCE.

### STATEMENTS AS TO THE RIGHTS TO THE INVENTION MADE UNDER FEDERALLY SPONSORED RESEARCH AND DEVELOPMENT

NONE

### BACKGROUND OF THE INVENTION

1. Field of the Invention

The present invention pertains to a method and apparatus for the production of bio-fuels and related substances including, but not limited to, ethanol. More particularly, the present invention pertains to a continuous flow microbial system that can be used in the production of bio-fuels and other substances.

2. Brief Description of the Prior Art

As demand for fossil fuel increases, and supply decreases, the costs associated with such fossil fuels can be significant. Additionally, many believe that consumption of fossil fuels negatively impacts the environment by contributing to global warming. Thus, an effort has been underway to find alternative energy sources to replace and/or supplement conventional fossil fuels.

Much attention has focused on bio-fuels as a possible alternative to conventional fossil fuels. Generally, bio-fuels are solid, liquid or gaseous fuels obtained from relatively recently lifeless or living biological material. By contrast, fossil fuels are fuels derived from long-dead biological material. One such bio-fuel that has received a great deal of attention is ethanol, a primarily plant-based fuel which can be produced from organic sources such as sugar cane, corn, waste paper. Ethanol can also be produced from grains like wheat or sorghum.

Ethanol, also known as ethyl alcohol, is a volatile, flammable, colorless liquid having a wide variety of uses including, but not limited to, as a fuel. For example, ethanol has a long history as a fuel for heat and light, and also as a fuel and/or fuel additive for internal combustion engines. When added to gasoline, ethanol reduces volatile organic compound and hydrocarbon emissions, carcinogenic benzene and butadiene emissions, and particulate matter emissions from internal combustion engines. Ethanol is also widely used as a solvent of substances intended for human contact or consumption.

Although ethanol can be produced via the hydration of ethylene, it is commonly produced biologically through culturing yeast under certain desired conditions in a process commonly referred to as fermentation. Conventional ethanol production systems utilizing fermentation frequently utilize “batch processing” methods, which require continuous flow to be interrupted for extended periods. As such, conventional fermentation plants typically include at least one large tank for storing materials for extended retention periods during different stages of the production process.

Feed stocks for the production of ethanol can include, but are not limited to, sugarcane juice, sugarcane syrup, molasses, bagasse, corn, fruit juice and concentrates, purified sugars such as sucrose, glucose, fructose, maltose, and syrup mixtures containing simple sugars such as those found in drink syrups. When certain species of yeast (for example, Saccharomyces cerevisiae) metabolize sugar, the yeast can produce ethanol and carbon dioxide. Ethanol can also be produced biologically from starchy materials such as cereal grains; however, in such cases, the starchy material must first be converted into sugar(s).

Existing processes for the production of ethanol (including, without limitation, fermentation systems) have proven to be inefficient and expensive, and frequently require large amounts of space. Thus, there is a need for a process that can be used in place of traditional fermentation systems, while reducing costs and space requirements, associated with conventional ethanol production processes. Further, there is a need for a method and apparatus for continuous flow production of bio-fuels including, without limitation, ethanol.

### SUMMARY OF THE PRESENT INVENTION

In the preferred embodiment, the present invention beneficially comprises at least one immobilized microbe bioreactor (“IMBR”) cluster having a plurality of IMBR microbial generation reactors, beneficially including oxygen source(s) for periodic oxygenation of such generation reactors. Such oxygen can be introduced as air pumped via conventional fans or air blowers, or as pure oxygen. It is to be observed that the system of the present invention can function as a stand-alone system for production of desired bio-fuels.

In the preferred embodiment, microbial generation and is accomplished using IMBR technology in which microbes are immobilized on a desired substrate. Such IMBR technology beneficially utilizes at least one bio-carrier medium inoculated with desired microbes; said at least one bio-carrier medium can include, without limitation, porous diatomaceous earth solids (such as described in U.S. Pat. No. 4,859,594 and U.S. Pat. No. 4,775,650, both of which are incorporated herein by reference). In the preferred embodiment, said at least one bio-carrier medium is beneficially coated with a thin film of chitin or other substance, and yeast cells or other beneficial microbes are immobilized on the surfaces of such at least one bio-carrier medium. Further, in the preferred embodiment, at least one micro bubble generator (MBG) immobilized cell reactor (for example, the MBG more fully disclosed in U.S. Pat. No. 5,534,143, which is incorporated herein by reference) is provided for periodic aeration and nutrient addition to a liquid column with bottom-up flow in certain of said IMBR reactor(s).

By promoting in-situ growth of yeast and/or other beneficial microbial populations, the present invention promotes microbial growth and acclimation within the fermentation tanks, piping and associated elements of the present invention. Over time, the microbial growth provided by the present invention can result in the spread of yeast and/or other beneficial microbial agents throughout the fermentation system, thereby improving the fermentation process and overall system efficiency.

Varying feed stocks can be used for fermentation including, but not limited to, sugars from cellulose and other materials, sugarcane juice, sugarcane syrup, molasses, fruit juice and concentrates, purified sugars such as sucrose, glucose, fructose, maltose, and syrup mixtures containing simple sugars such as those found in drinks syrups.

In the preferred embodiment, such feed stocks are beneficially tested for initial concentrations. Feed stocks falling within desired ranges (such as, for example, between 15 and 30 degrees BRIX [° Bx]) can be directly introduced with nutrient amendment into the system. Feed stocks having higher concentrations can be diluted to meet desired specifications.

Make up water is provided from clean or recycled sources. Makeup water, nutrients (such as nitrogen, sulfur, and/or phosphorus containing compounds) and/or antibiotics can be added to the feed stream prior to reactor injection. The pre-fermentation tanks can also beneficially utilize mixing to ensure a homogeneous feed for reactor injection.

Following pre-fermentation stage, treated feed stocks are sent to a fermentation stage. Flow rates through the reactors can be adjusted based upon volume desired. Reactor sizes can be varied and the void volume within the reactors either filled or partially filled with microbially inoculated bio-carrier media. In the preferred embodiment, off gases are recaptured using a vacuum system which then returns gases to the MBG.

IMBR technology can be beneficially used to completely replace traditional submerged tank fermentation processes. By increasing microbial contact with feedstock materials using concentrated microbial populations permanently attached to bio-carrier media, the present invention permits continuous flow production of bio-fuels including, without limitation, ethanol. Moreover, the present invention eliminates the need for prolonged storage or retention times common with conventional batch-type production methods, and eliminates the need for large storage tanks common with conventional production methods. The present invention also allows for total control of material flow, nutrient addition, and oxygenation using a specific aeration protocol for timed anaerobiosis, thereby allowing for increased and consistent alternative fuels production from feed stocks.

It is to be observed that the reactors of the present invention can be arranged in any number of different arrangements. By way of illustration, but not limitation, such reactors can be arranged in a cluster of five (5) IMBR reactors, wherein the first four (4) reactors can be arranged for feeding—in parallel or in series—followed by final polishing for streams from all four (4) feeding reactors by the fifth (5^{th}) reactor.

When five IMBR reactors are utilized, the first four (4) reactors may be oxygenated for a variable periods of time in each 6 or longer hours of continuous flow production. Further, anaerobic conditions can be maintained in the final reactor, allowing for constant anaerobic conditions for final polishing and alternative fuels production from feed stocks. This process further provides for reseeding of the final reactor from the flow streams of other reactors, as excess microbes are carried to the final reactor via such flow streams.

Time for alternative fuels production is substantially reduced as the flow through the system is continuous without need for cleanout of fermentation/production tanks as required in traditional fermentation systems using batch production. The footprint of a production facility utilizing the present invention is significantly reduced as no large tanks for batch fermentation are required.

Although primarily described herein in connection with the production of bio-fuels, ethanol and related substances, it is to be observed that the method and apparatus of the present invention can further utilize food grade materials, and control is appropriate for the generation of microbes and food-related materials they produce, such as the fermentation of beer, wine, cheese, yogurt, and other similar food products. The present system can also use medical grade materials, and control is appropriate for the generation of medical related microbes and the materials they manipulate or generate.

The following testing data is illustrative of the method and apparatus of the present invention, and is not to be limiting in any way:

Data Set 1:

These data were produced using an immobilized yeast culture of Saccharomyces cerevisiae. Cell growth in preliminary tests indicated 10⁹ cells/g biocarrier or greater was achievable in a 3% ethanol broth. Main sugar source within broth was either purified sucrose and micronutrients or molasses and micronutrients. Table 1/FIG. 5 present data on continuous feed with a 24 hour residence time. Benchscale IMBR reactors with an 1800 ml void volume were run in series, that is the entire flow passed into the first reactor and then into the second reactor, before the material was collected for distillation. Aeration periodically was provided by a low flow air source on a timer to allow for appropriate oxygenation to promote cellular growth. Samples were taken from feed to monitor initial Total Reducing Sugars in fermented medium (TRSf) and after fermentation to determine residual sugar content. A refractometer was used to read initial and final sugar content. Ethanol production was determined via mass distillation and recovery.

As the microcolumn population acclimated to the presence of ethanol, the overall alcohol content/production increased until a maximum production of ˜70 g/L was detected at 72 hours continuous flow and feeding. For continuous flow fermentation, D is a ratio, expressed as/hour, and calculated by dividing the medium flow rate (F) by the working volume of the reactor vessel (V). Flow was 7.5 ml/min into an 1800 ml void volume single reactor. This was a 4 hour (hydraulic retention time) HRT per reactor or 8.33 HRT for both IMBRs in series, plus post reactor plumbing and holding having a total of 24 hours retention time for yield at equivalent degrees Brix or TRSf of our system. Feed and ethanol liquor export sugar concentrations are reported as Total Reducing Sugars in fermentable material (TRSf) (Table 1, FIG. 5). Feed concentrations were generally maintained at ˜160 g/L. By 9 hours continuous flow, concentrations of ethanol recovered were 45 g/L outflow. After several cycles of aeration and anaerobiosis, ethanol recovery had increased to ˜70 g/L outflow. TRSf concentrations decreased appropriately over time.

TABLE 1
Continuous Fermentation using Dual Column IMBR system
IMBR Feed: 0.25/h Retention Time: 24 hours

| | Feed | Export | Ethanol Yield |
|---|---|---|---|
| Time (hours) | TRSf (g/L) | TRSf (g/L) | (g/L) |
| 0 | 160 | 160 | |
| 3 | 162 | 108 | |
| 6 | 158 | 101 | |
| 9 | 161 | 77 | 45 |
| 12 | 160 | 72 | 46 |
| 15 | 157 | 56 | 48 |
| 22 | 162 | 48 | 51 |
| 28 | 158 | 42 | 52 |
| 32 | 155 | 37 | 55 |
| 39 | 159 | 22 | 61 |
| 42 | 157 | 18 | 65 |
| 48 | 158 | 12 | 64 |
| 52 | 156 | 11 | 68 |
| 60 | 160 | 12 | 69 |
| 72 | 160 | 9 | 71 |

After column acclimation, feed concentrations were increased and maintained above 200 g/L TRSf. Ethanol yields beginning at 9 hours were found to be above 50 g/L achieving a sustained maximum of 70 g/L by 32 hours. Sampling continued for 110 hours (Table 2, FIG. 6). Ethanol concentrations continued to be high throughout the remaining sample period.

TABLE 2
Continuous Fermentation after acclimation (Dual Column)
IMBR Feed: 0.25/h Total Retention Time: 24 hours

| | Feed | Export | Ethanol Yield |
|---|---|---|---|
| Time (hours) | TRSf (g/L) | TRSf (g/L) | (g/L) |
| 0 | 220 | 16 | |
| 3 | 220 | 14 | |
| 6 | 210 | 22 | |
| 9 | 208 | 17 | 55 |
| 12 | 222 | 27 | 54 |
| 15 | 214 | 21 | 51 |
| 22 | 210 | 28 | 55 |
| 28 | 209 | 12 | 68 |
| 32 | 220 | 14 | 69 |
| 39 | 220 | 22 | 71 |
| 42 | 218 | 18 | 72 |
| 48 | 219 | 12 | 70 |
| 52 | 217 | 11 | 68 |
| 60 | 217 | 12 | 69 |
| 72 | 219 | 9 | 71 |
| 96 | 220 | 9 | 70 |
| 110 | 218 | 9 | 70 |

A larger benchscale reactor system having a void volume of 4.0 L, was utilized for testing optimization conditions for a larger scale system. Concentrations of the feed material were varied to determine optimum loading rates for full scale fermentation. A 96 hour window of sampling and testing was used for each concentration to allow for reactor acclimation. (Table 3, FIG. 7).

TABLE 3
Continuous Fermentation at different loading rates (Dual Column)
IMBR Feed: 0.25/h Retention Time: 24 hours

| Time | Feed | Export | Ethanol Yield #1 |
|---|---|---|---|
| (hours) | TRSf (g/L) | TRSf (g/L) | (g/L) |
| 0 | 220 | 16 | 47 |
| 6 | 220 | 14 | 53 |
| 24 | 210 | 22 | 51 |
| 48 | 208 | 17 | 55 |
| 96 | 210 | 17 | 55 |
| 0 | 300 | 21 | 51 |
| 6 | 305 | 28 | 55 |
| 24 | 301 | 29 | 68 |
| 48 | 310 | 36 | 69 |
| 96 | 307 | 38 | 71 |
| 0 | 420 | 39 | 48 |
| 6 | 420 | 57 | 45 |
| 24 | 418 | 61 | 44 |
| 48 | 417 | 66 | 46 |
| 96 | 420 | 68 | 41 |
| 0 | 500 | 114 | 46 |
| 6 | 500 | 122 | 41 |
| 24 | 501 | 154 | 39 |
| 48 | 505 | 136 | 38 |
| 96 | 501 | 127 | 41 |

Thus, it is an object of the present invention to provide a process for permanent immobilization of microbes (such as yeast, Saccharomyces cerevisiae, or bacterial consortia having one or more beneficial organisms) on a substrate for the purpose of acting on carbohydrate based feed stocks to produce bio-fuels and/or other alternative fuels including, but not limited to, ethanol, butanol, methanol, biodiesel and others, as well as food and medical grade materials produced through fermentation. Such process beneficially increases the population of such organisms allowing for highly concentrated and consistent populations throughout the production system.

It is a further object of the present invention to utilize an ultra efficient aeration system, such as one or more immobilized microbe bioreactors, to enhance growth and stability of microbial populations through cycling of aerobic and anaerobic conditions during selected time periods. The flow through an IMBR cluster can be tailored to accommodate feed stocks so that each IMBR within a cluster can run in parallel, or in series, as desired.

The system of the present invention, when using food grade materials and control, is appropriate for the generation of microbes for use in food related fermentation such as the production of beer, wine, cheese, yogurt, and other fermented food products. Likewise, the system of the present invention, when using medical grade materials and control, is appropriate for the generation of medical related microbes and the materials they manipulate or generate.

### BRIEF DESCRIPTION OF THE DRAWINGS

The foregoing summary, as well as the following detailed description of the preferred embodiments, is better understood when read in conjunction with the appended drawings. For the purpose of illustrating the invention, the drawings show certain preferred embodiments. It is understood, however, that the invention is not limited to the specific methods and devices disclosed.

FIG. 1 depicts a schematic layout of a pre-fermentation stage of the continuous flow ethanol production system of the present invention.

FIG. 2 depicts a schematic layout of a fermentation stage of the continuous flow ethanol production system of the present invention.

FIG. 3 depicts a schematic layout of a distillation stage of the continuous flow ethanol production system of the present invention.

FIG. 4 depicts a schematic layout of a continuation of the distillation stage of the continuous flow ethanol production system of the present invention.

FIG. 5 depicts a graphical representation of data reflecting continuous fermentation with an IMBR with comparison to commercial yield.

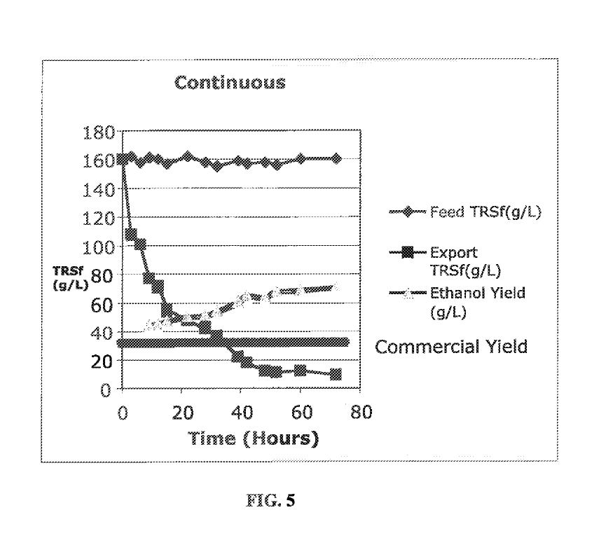

FIG. 6 depicts a graphical representation of continuous fermentation after acclimation.

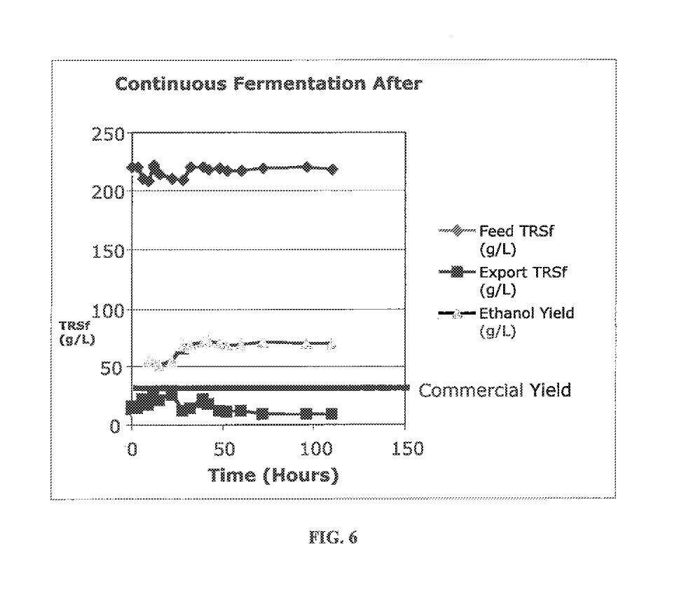

FIG. 7 depicts a graphical representation of loss of ethanol yield as feed concentration increases (nitrogen is limiting).

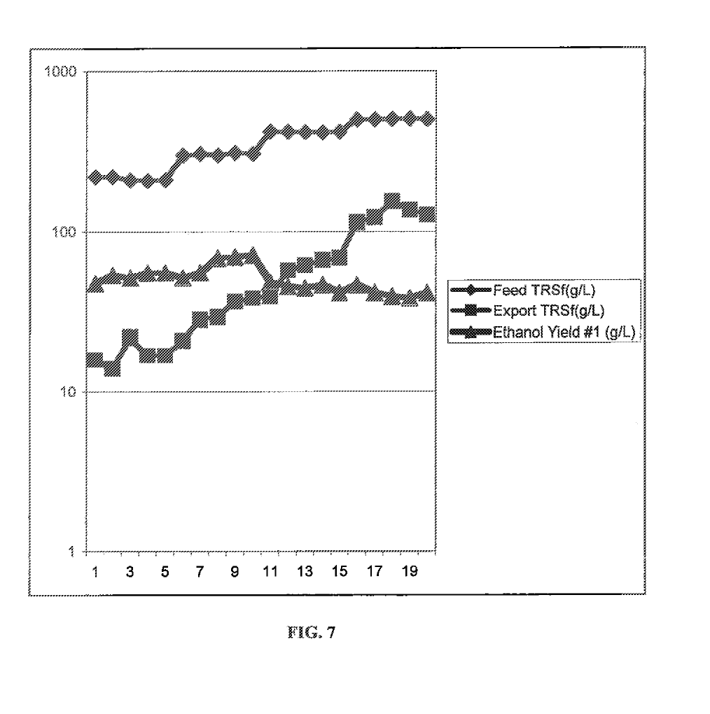

### DETAILED DESCRIPTION OF A PREFERRED EMBODIMENT OF THE INVENTION

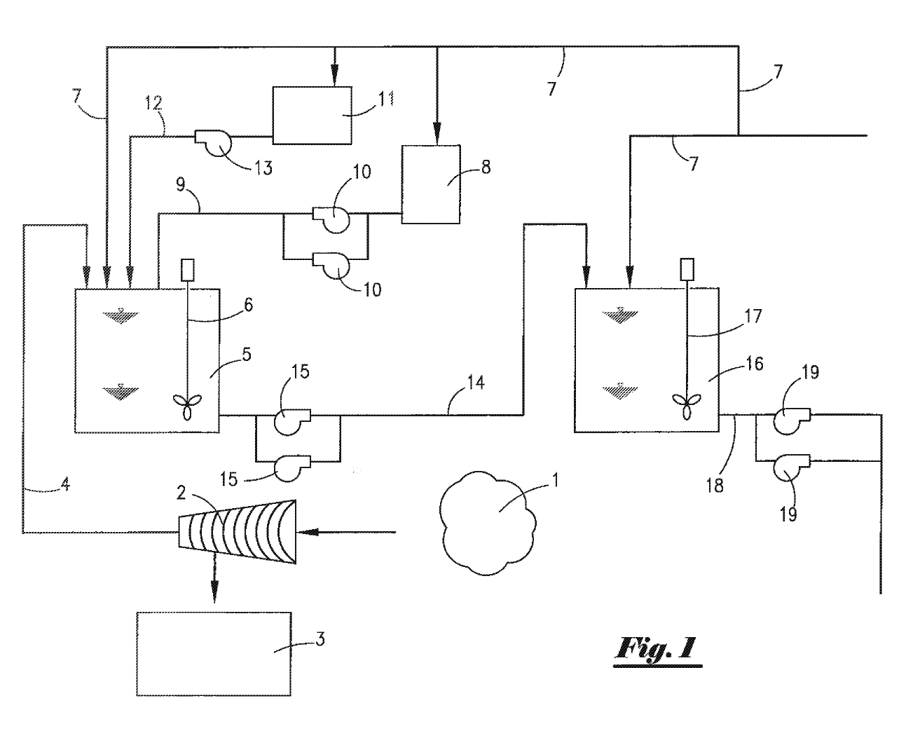

Referring to the drawings, FIG. 1 depicts a pre-fermentation stage of the continuous flow production system of the present invention. Although many different feed stocks can be used in connection with the present invention, in the preferred embodiment, such feed stocks can include but are not limited to sugars from cellulose and other materials, sugarcane juice, sugarcane syrup, molasses, fruit juice and concentrates, purified sugars such as sucrose, glucose, fructose, maltose, and syrup mixtures containing simple sugars such as those found in drinks syrups.

In the preferred embodiment, feed stocks are tested for initial concentrations. Ranges between 15 and 30 degrees Brix (° Bx) can be directly introduced with nutrient amendment into the system. Higher concentrated feeds can be diluted to appropriate concentrations.

In the preferred embodiment, feed stock may include one or more solids. Accordingly, in the preferred embodiment raw feed stock 1 enters said pre-fermentation stage via solids separation centrifuge 2. Solids can be directed to solids collection vessel 3, with liquid feed stock proceeding via line 4 to pre-fermentation nutrient amendment/antibiotic tank 5 having at least one mixer 6. Make-up water can be provided to pre-fermentation nutrient amendment/antibiotic tank 5 using water supply line 7. Make-up water can be provided from outside sources, or from recycled sources within the distillation component of the production system discussed below.

In addition to make-up water, nutrients (such as, for example, nitrogen, sulfur, and/or phosphorus-containing compounds) as well as antibiotics, can be beneficially added to the feed stream prior to delivery to the fermentation stage of the present invention. Specifically, in the preferred embodiment, nutrients can be added to liquid feed stock in pre-fermentation nutrient amendment/antibiotic tank 5 from nutrient supply tank 8 via line 9 using at least one metering pump 10. Similarly, antibiotics can be added to liquid feed stock in pre-fermentation nutrient amendment/antibiotic tank 5 from antibiotic supply tank 11 via line 12 using at least one metering pump 13.

Treated liquid feed stock is transferred out of pre-fermentation nutrient amendment/antibiotic tank 5 and into pre-fermentation holding tank 16 via line 14 using at least one transfer pump 15. In the preferred embodiment, pre-fermentation holding tank 16 is equipped with at least one mixer 17. If desired, make-up water can also be supplied to pre-fermentation holding tank 16 via make-up water line 7.

In the preferred embodiment, mixing is continuous in both pre-fermentation holding tank 16 and pre-fermentation mixing tank 5 to maintain desired Brix values. Automatic sampling and testing of feed stock concentration can be used to monitor concentration and determine make-up water volume to be added to such feed stock. In the preferred embodiment, automatic nutrient addition can occur as needed based upon Brix values, while antibiotics can also be added based upon measured values of feed stock concentration. Feed stock can then be directed from pre-fermentation holding tank 16 to a fermentation phase of the present invention via line 18 using at least one transfer pump 19. In the preferred embodiment, prepared feed to the reactor systems can be maintained for desired periods prior to fermentation stage of the present invention.

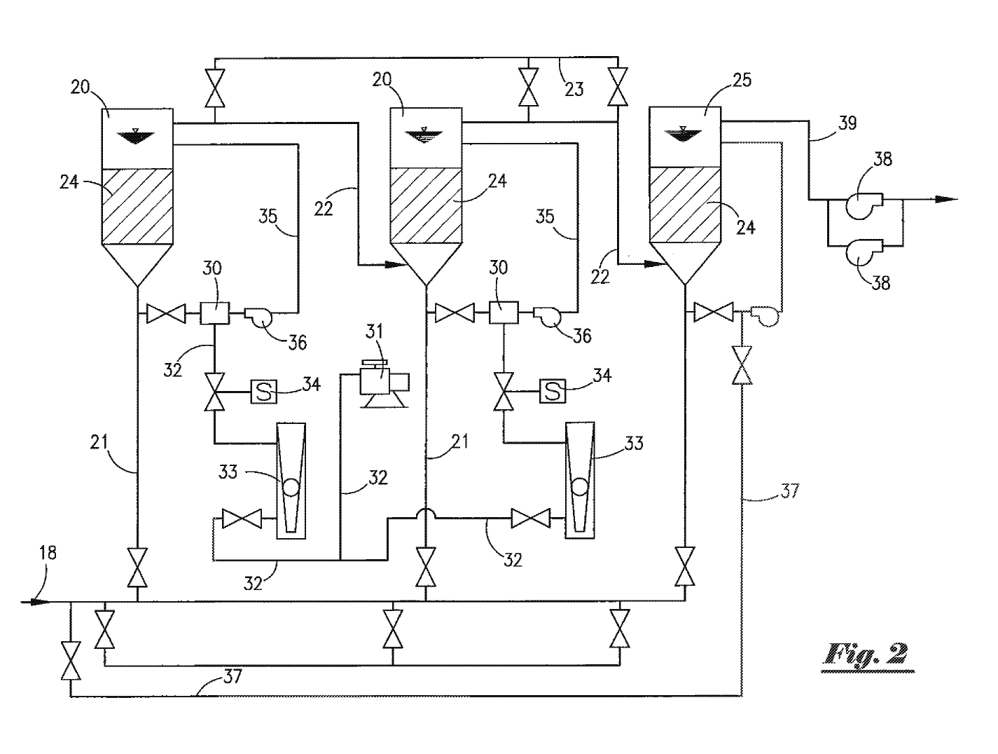

FIG. 2 depicts a fermentation stage of the continuous flow production system of the present invention. In the preferred embodiment, the present invention utilizes a plurality of IMBR reactors arranged in a cluster for microbial generation in such fermentation stage. As set forth above, such IMBR technology beneficially utilizes at least one bio-carrier medium inoculated with desired microbes; said at least one bio-carrier medium can include, without limitation, porous diatomaceous earth solids (such as described in U.S. Pat. No. 4,859,594 and U.S. Pat. No. 4,775,650, which are incorporated herein by reference). By promoting in-situ growth of desired yeast and/or other microbial populations, the present invention promotes microbial growth and acclimation within the fermentation tanks, piping and associated elements of the present invention. Over time, the microbial growth provided by the present invention can result in the spread of yeast and/or other beneficial microbial agents, thereby improving the fermentation process and overall system efficiency.

As reflected in FIG. 2, amended feed stock is provided to the fermentation stage via line 18. Said amended feed stock enters IMBR reactors 20 at the bottom of such reactors via flow lines 21; in the preferred embodiment, a split flow supplies such amended feed stock to reactors 20. In the preferred embodiment, such reactors are arranged into two separate groups comprising at least one “feeding” reactor 20, and at least one “polishing” reactor 25.

It is to be observed that flow through multiple reactors can be configured in many different ways that are too numerous to identify herein. For example, reactors 20 can be run in series with feed stock moving from a first reactor 20 to one or more other reactor(s) 20 in series (via series effluent line 22) before ultimately reaching polishing reactor 25. Alternatively, reactors 20 can also be run strictly in parallel where each reactor 20, or isolated groups of reactors 20, is individually supplied with feedstock material, and effluent from such reactors 20 is then provided directly to polishing reactor 25 via parallel effluent line 23. Moreover, if desired, reactors 20 and/or 25 can be grouped or arranged in multiple other combinations.

Referring to the arrangement depicted in FIG. 2, amended feed stock enters the bottom of reactors 20 via flow lines 21. In the preferred embodiment, oxygenation of reactors 20 occurs for limited time periods at beneficially determined intervals. In the preferred embodiment, an electronically monitored and controlled aeration protocol is used to maintain appropriate oxygenation to speed microbial growth without loss of production capacity and/or microbial acclimation. Oxygen sensors can be used to detect oxygen levels within outflow of reactors 20 during the aeration cycle, and adjust oxygen supply accordingly. It is to be observed that aeration protocols will vary with reactor flow protocols to maintain appropriate microbial growth and product (for example, ethanol) tolerance depending upon reactor position in flow protocols.

In the preferred embodiment, at least one micro bubble generator 30 (for example, the MBG more fully disclosed in U.S. Pat. No. 5,534,143, which is incorporated herein by reference) is provided for periodic aeration and nutrient addition in reactors 20 using bottom-up flow. Oxygen from air or other oxygen source is provided to MBG 30. In the preferred embodiment, such oxygen is supplied by air compressor 31 via air lines 32, each of which is in turn equipped with rotometer 33 and solenoid valve 34. In the preferred embodiment, reactors 20 are further equipped with recirculation lines 35 and recirculation pumps 36 for capturing and re-circulating gasses from said reactors 20.

Reactors 20 and 25 are beneficially loaded with desired quantities of at least one inoculated bio-carrier medium 24. Said at least one bio-carrier medium 24 is beneficially inoculated with desired microbes and/or other cultures; said at least one bio-carrier medium 24 can include, without limitation, porous diatomaceous earth solids (such as described in U.S. Pat. No. 4,859,594 and U.S. Pat. No. 4,775,650, which are incorporated herein by reference). In the preferred embodiment, said at least one bio-carrier medium 24 is beneficially coated with a thin film of chitin or other substance, and yeast cells or other beneficial microbes are immobilized on the surfaces of such at least one bio-carrier medium 24.

In the preferred embodiment, oxygenation of reactors 20 provided by MBG 30 is electronically controlled. In many cases, anaerobic conditions are encouraged within reactors 20 during periods when oxygenation is not occurring, while anaerobic conditions are maintained continuously in reactor 25. Oxygenation of reactors 20 allows for enhanced growth of microbial cells and removal of built up residual materials within reactors 20. Polishing of any remaining feed stock material leaving reactors 20 can then be performed in reactor 25 utilizing excess microbes arriving with outflow from reactors 20. In the preferred embodiment, microbial concentration can be periodically monitored to ensure constant and concentrated populations to maintain consistent production performance, and samples can be taken from multiple locations within the reactors. Effluent from reactor 25 is piped via line 39 using transfer pumps 38.

Flow rates through the reactors can be tailored based upon starting concentrations of feed stock. Reactor sizes can be varied and the void volume within such reactors can be either filled or partially filled with at least one inoculated bio-carrier media 24. In the preferred embodiment, fermentation seed line 37 is provided for optional routing of seeding materials from reactor 25 to the inlet of reactors 20. Additionally, a surge tank (not shown in the drawings) may be provided for collection of effluent from reactor 25 to ensure consistent flow of such effluent to downstream equipment, such as a recovery distillation system. Efficiency increases as the system becomes more tolerant of alcohol content.

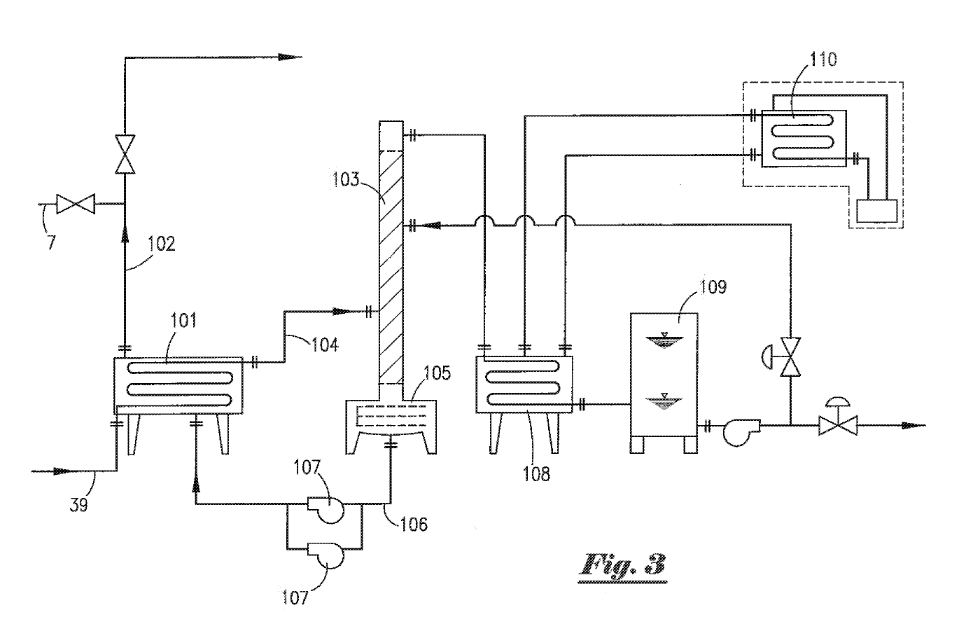

FIG. 3 depicts a schematic layout of a distillation stage of the continuous flow ethanol production system of the present invention. Effluent leaving fermentation stage of the present invention (depicted in FIG. 3) via line 39 can constitute a number of different beneficial or desired substances depending on the configuration of the present invention. By way of illustration, but not limitation, it is to be observed that said effluent may constitute beer or liquor produced during the fermentation stage.

Effluent entering the distillation stage of the present invention is directed via line 39 to heat exchanger 101. Water leaving heat exchanger 101 can be sent for disposal via line 102, or reused as make-up water via line 7. Effluent from said heat exchanger can be sent to distillation column 103 via line 104. Boiler 105 is provided for distillation column 103, and also can be used to supply heat to heat exchanger 101 via line 106 having transfer pumps 107. If desired, the distillation process depicted in FIG. 3 can also include ethanol condenser 108, reflux tank and chill water unit 110, as well as associated transfer piping depicted in FIG. 3.

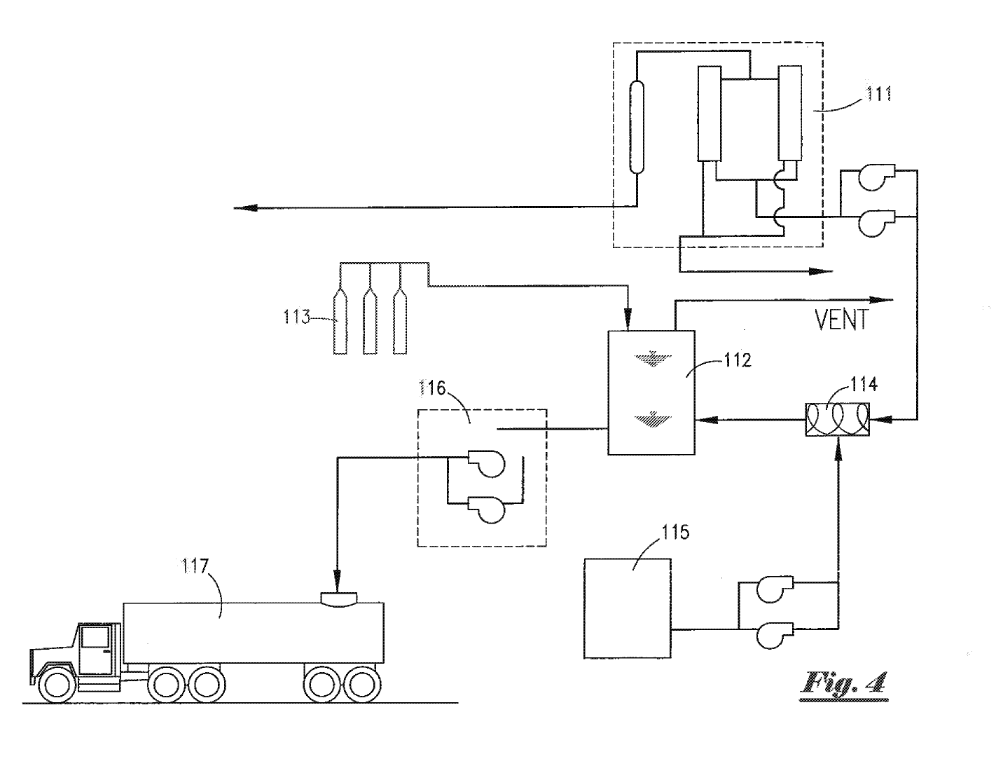

FIG. 4 depicts a schematic layout of a continuation of the distillation stage of the continuous flow ethanol production system of the present invention. Effluent product leaving the distillation components depicted in FIG. 3 can be further processed using molecular sieve 111, vented anhydrous ethanol storage tank 112, nitrogen head gas supply 113, static mixer 114 and denature chemical tank 115, along with associated transfer piping depicted in FIG. 4. In the preferred embodiment, loading station 116 is provided for transfer of finished product to haul tanker 117 for transportation to sale or ultimate disposition.

It is to be observed that the various components and arrangement of the distillation system(s) depicted in FIGS. 3 and 4 are for illustration only. Such distillation systems may contain other components known in the art, or may not include certain components depicted in such drawings, without departing from the scope of the present invention.

The above-described invention has a number of particular features that should preferably be employed in combination, although each is useful separately without departure from the scope of the invention. While the preferred embodiment of the present invention is shown and described herein, it will be understood that the invention may be embodied otherwise than herein specifically illustrated or described, and that certain changes in form and arrangement of parts and the specific manner of practicing the invention may be made within the underlying idea or principles of the invention.

What is claimed:

1. A method of producing bio-fuel comprising:
a. providing feed stock into a pre-fermentation system, wherein said pre-fermentation system comprises:
i. a mixing tank;
ii. a nutrient source; and
iii. an antibiotic source;
b. measuring the sugar concentration of said feed stock;
c. adjusting said sugar concentration of said feed stock to fall within a range between 15 and 30 degrees BRIX (° Bx);
d. directing amended feed stock from said pre-fermentation system to a fermentation system, wherein said fermentation system comprises:
i. a first bio-reactor having an interior and comprising at least one bio-carrier medium disposed within said first bio-reactor, wherein at least one microorganism, preselected for fermentation capability, is immobilized on the surface of said at least one bio-carrier medium using chitin, and said at least one microorganism propagates on the surface of said at least one bio-carrier medium and spreads throughout said interior of said first bio-reactor;
ii. a second bio-reactor having an interior and comprising at least one bio-carrier medium disposed within said second bio-reactor, wherein second bioreactor is connected in series with said first bio-reactor and at least one microorganism, preselected for fermentation capability, is immobilized on the surface of said at least one bio-carrier medium using chitin, said at least one microorganism propagates on the surface of said at least one bio-carrier medium and spreads throughout said second bio-reactor, and said second bio-reactor is maintained in a substantially constant anaerobic condition;
iii. a-micro bubble generator for providing oxygen to said first bio-reactor;
e. providing oxygen to said first bio-reactor on a periodic basis to alternate said first bio-reactor between substantially aerobic and substantially anaerobic conditions;
f. generating effluent from said fermentation system; and
g. distilling said effluent from said fermentation system to generate biofuel.
